(19) United States
(12) Patent Application Publication
(10) Pub. No.: US 2001/0021375 A1
(43) Pub. Date: Sep. 13, 2001

(54) Cosmetic or dermatological sunscreen preparations

(76) Inventors: Peter Hossel, Schifferstadt (DE); Thomas Wunsch, Speyer (DE); Reinhold Dieing, Schifferstadt (DE)

Correspondence Address: Herbert B. Keil, KEIL & WEINKAUF, 1101 Connecticut Ave., N.W., Washington, DC, 20036, US

(21) Appl. No.: 09/771,595

(22) Filed: Jan. 30, 2001

(30) Foreign Application Priority Data
Feb. 18, 2000 (DE) 10007486.3

(51) Int. Cl. A61K 7/42

(52) U.S. Cl. 424/59

(57) ABSTRACT

The invention relates to mixtures comprising

A) at least one copolymer obtainable by

(i) free-radically initiated solution polymerization of a monomer mixture of

(a) 0.01 to 99.99% by weight of at least one monomer chosen from the group consisting of N-vinylimidazoles and diallylamines, optionally in partially or completely quaternized form;

(b) 0.01 to 99.99% by weight of at least one neutral or basic water-soluble monomer which is different from (a);

(c) 0 to 50% by weight of at least one unsaturated acid or an unsaturated anhydride;

(d) 0 to 50% by weight of at least one free-radically copolymerizable monomer which is different from (a), (b) and (c); and

(e) 0 to 10% by weight of at least one monomer having at least two ethylenically unsaturated nonconjugated double bonds which acts as crosslinker, and

(ii) subsequent partial or complete quaternization or protonation of the polymer where the monomer (a) is not quaternized or only partially quaternized and

B) at least one inorganic UV filter

and to the use thereof for the preparation of cosmetic or dermatological light protection agent preparations.

The present invention relates to a mixture of at least one copolymer obtainable by means of solution polymerization, and at least one inorganic UV filter, and to the use thereof for the preparation of cosmetic and dermatological sunscreen preparations.

The sunscreens used in cosmetic and dermatological preparations have the task of preventing or at least diminishing the extent of the harmful effects of sunlight on human skin. In addition, these sunscreens, however, also serve to protect other ingredients from decomposition or degradation by UV radiation. In hair cosmetic formulations, the aim is to prevent damage to the keratin fibers by UV rays.

The sunlight which reaches the Earth's surface has a proportion of UV-B radiation (280 to 320 nm) and of UV-A radiation (>320 nm), which directly border the visible light region. The effect on human skin is evident, particularly in the case of UV-B radiation, from sunburn. Accordingly, the industry offers a relatively large number of substances which absorb both UV-A radiation and UV-B radiation.

In this connection, in recent years, as well as organic UV absorbers, inorganic sunscreens have also become increasingly important in cosmetics and dermatology.

Suitable inorganic sunscreen filters which may be mentioned here are titanium dioxide, zinc oxide, iron oxides and also cerium oxide.

Micropigments, primarily micronized titanium dioxide or zinc oxide, are notable for their high compatibility and their particular stability. Extremely effective protection over a wide UV range from 250 to 380 nm can be achieved with titanium dioxides and/or zinc oxides.

However, the abovementioned inorganic filters have the disadvantage that, when used for the preparation of cosmetic or dermatological preparations, dispersion problems frequently arise since the particles often settle in the cosmetic formulations, meaning that optimum application to the skin is not guaranteed.

It is an object of the present invention to provide new types of cosmetic compositions for protecting the skin which have improved stability and good formulation properties, and moreover improved sensory properties and a high sun protection factor.

We have found that this object is achieved by mixtures comprising

A) at least one copolymer obtainable by

(i) free-radically initiated solution polymerization of a monomer mixture of

(a) 0.01 to 99.99% by weight, preferably 2 to 94.98% by weight, particularly preferably 10 to 70% by weight, of at least one monomer chosen from the group consisting of N-vinylimidazoles and diallylamines, optionally in partially or completely quaternized form;

(b) 0.01 to 99.99% by weight, preferably 5 to 97.98% by weight, particularly preferably 20 to 89.95% by weight, of at least one neutral or basic water-soluble monomer which is different from (a);

(c) 0 to 50% by weight, preferably 0 to 40% by weight, particularly preferably 0 to 30% by weight, of at least one unsaturated acid or an unsaturated anhydride;

(d) 0 to 50% by weight, preferably 0 to 40% by weight, particularly preferably 0 to 30% by weight, of at least one further free-radically copolymerizable monomer which is different from (a), (b) and (c); and

(e) 0 to 10% by weight, preferably 0.01 to 10% by weight, particularly preferably 0.02 to 8% by weight, very particularly preferably 0.05 to 5% by weight, of at least one monomer having at least two ethylenically unsaturated nonconjugated double bonds which acts as crosslinker, and

(ii) subsequent partial or complete quaternization or protonation of the polymer where the monomer (a) is not quaternized or only partially quaternized and

B) at least one inorganic UV filter.

Cationic polymers are already frequently used as conditioners in hair cosmetic formulations. They primarily improve the wet combability of hair. In addition, cationic polymers prevent electrostatic charging of the hair.

Thus, for example in EP-A-0 246 580, the use of noncrosslinked homo- and copolymers of 3-methyl-1-vinylimidazolium chlorides in cosmetic compositions is described. EP-A-0 544 158 and U.S. Pat. No. 4,859,756 claim the use of noncrosslinked homo- and copolymers of chloride-free quaternized N-vinylimidazoles in cosmetic preparations. EP-A-0 715 843 discloses the use of noncrosslinked copolymers of a quaternized N-vinylimidazole, N-vinylcaprolactam and N-vinylpyrrolidone, and optionally a further comonomer in cosmetic preparations.

EP-A-0 893 117 describes the use of crosslinked cationic copolymers as conditioners in preparations for hair cosmetics.

Component A) present in the mixtures according to the invention can either be noncrosslinked or crosslinked cationic polymers. Within the scope of the present invention, preference is given to crosslinked cationic copolymers.

Suitable monomers (a) are the N-vinylimidazole derivatives of the formula (I)

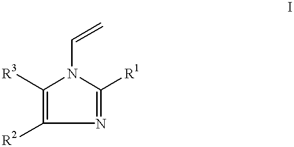

in which the radicals R¹ to R³, independently of one another, are hydrogen, C₁-C₄-alkyl or phenyl.

Also suitable are diallylamines of the formula (II)

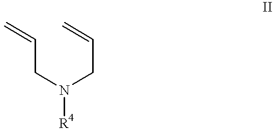

in which R⁴ is C₁-C₂₄-alkyl.

Examples of compounds of the formula (I) are given in Table 1 below:

TABLE 1

| R¹ | R² | R³ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

Other monomers of the formula (I) which can be used are the ethyl, propyl or butyl analogs of the methyl-substituted 1-vinylimidazoles listed in Table 1.

Examples of compounds of the formula (II) are diallylamines in which R⁴ is methyl, ethyl, iso- or n-propyl, iso-, n- or tert-butyl, pentyl, hexyl, heptyl, octyl, nonyl or decyl. Examples of longer-chain radicals R⁴ are undecyl, dodecyl, tridecyl, pentadecyl, octadecyl and icosyl.

The monomers (a) can either be used in quaternized form as monomers or be polymerized in non-quaternized form, in the latter case the resulting copolymer being either quaternized or protonated.

Suitable for the quaternization of compounds of the formulae (I) and (II) are, for example, alkyl halides having 1 to 24 carbon atoms in the alkyl group, e.g. methyl chloride, methyl bromide, methyl iodide, ethyl chloride, ethyl bromide, propyl chloride, hexyl chloride, dodecyl chloride, lauryl chloride and benzyl halide, in particular benzyl chloride and benzyl bromide. Other suitable quaternizing agents are dialkyl sulfates, in particular dimethyl sulfate or diethyl sulfate. The quaternization of the basic monomers of the formulae (I) and (II) can also be carried out with alkylene oxides, such as ethylene oxide or propylene oxide, in the presence of acids.

The quaternization of the monomer or of a polymer with one of said quaternizing agents can be carried out by generally known methods.

The copolymer can be quaternized completely or else only partially. The proportion of quaternized monomers (a) in the copolymer can vary over a wide range and is, for example, from about 20 to 100 mol %.

Preferred quaternizing agents are methyl chloride, dimethyl sulfate or diethyl sulfate.

Preferred examples of monomers (a) are 3-methyl-1-vinylimidazolium chloride and methosulfate, dimethyldiallylammonium chloride.

Particularly preferred monomers (a) are 3-methyl-1-vinylimidazolium chloride and methosulfate.

Suitable for the protonation are, for example, mineral acids, such as HCl, H₂SO₄, H₃PO₄, and monocarboxylic acids, such as, for example, formic acid and acetic acid, dicarboxylic acids and polyfunctional carboxylic acids, such as, for example, oxalic acid and citric acid, and all other proton-donating compounds and substances which are able to protonate the corresponding vinylimidazole or diallylamine. Water-soluble acids are particularly suitable for the protonation.

The protonation of the polymer can either be carried out after the polymerization or during the preparation of the mixture, during which a physiologically compatible pH is usually set.

The term “protonation” means that at least some of the protonatable groups of the polymer, preferably 20 to 100 mol %, is protonated, resulting in a cationic overall charge of the polymer.

Suitable monomers (b) which are different from (a) are N-vinyllactams, such as, for example, N-vinylpiperidone, N-vinylpyrrolidone and N-vinylcaprolactam, N-vinylacetamide, N-methyl-N-vinylacetamide, acrylamide, methacrylamide, N,N-dimethylacrylamide, N-methylolmethacrylamide, N-vinyloxazolidone, N-vinyltriazole, hydroxyalkyl (meth)acrylates, such as, for example, hydroxyethyl (meth)acrylate and hydroxypropyl (meth)acrylates, or alkyl ethylene glycol (meth)acrylates having 1 to 50 ethylene glycol units in the molecule. Also suitable are dialkylaminoalkyl (meth)acrylates and dialkylaminoalkyl(meth)acrylamides, such as, for example, N,N′-dimethylaminoethyl methacrylate or N-[3-(dimethylamino)propyl]methacrylamide.

Preference is given to using N-vinyllactams as monomers (b). Very particular preference is given to N-vinylpyrrolidone.

Suitable monomers (c) are unsaturated carboxylic acids and unsaturated anhydrides, such as, for example, acrylic acid, methacrylic acid, crotonic acid, itaconic acid, maleic acid, fumaric acid or their corresponding anhydrides, unsaturated sulfonic acids, for example acrylamidomethylpropanesulfonic acid, and the salts of the unsaturated acids, such as, for example, the alkali metal or ammonium salts.

Suitable monomers (d) are Cₗ-C₄₀-alkylesters of (meth)acrylic acid, where the esters are derived from linear, branched-chain or carbocyclic alcohols, e.g. methyl (meth)acrylate, ethyl (meth)acrylate, tert-butyl (meth)acrylate, isobutyl (meth)acrylate, n-butyl (meth)acrylate, stearyl (meth)acrylate, or esters of alkoxylated fatty alcohols, e.g. C₁-C₄₀-fatty alcohols, reacted with ethylene oxide, propylene oxide or butylene oxide, in particular C₁₀-C₁₈-fatty alcohols, reacted with 3 to 150 ethylene oxide units. Also suitable are N-alkyl-substituted acrylamides having linear, branched-chain or carbocyclic alkyl radicals, such as N-tert-butylacrylamide, N-butylacrylamide, N-octylacrylamide, N-tert-octylacrylamide.

Also suitable are styrene, vinyl esters and allyl esters of C₁l-C₄₀-carboxylic acids, which may be linear, branched-chain or carbocyclic, e.g. vinyl acetate, vinyl propionate, vinyl neononanoate, vinyl neoundecanoic acid, vinyl t-butylbenzoate, alkyl vinyl ethers, for example methyl vinyl ether, ethyl vinyl ether, butyl vinyl ether, stearyl vinyl ether.

Acrylamides, such as N-tert-butylacrylamide, N-butylacrylamide, N-octylacrylamide, N-tert-octylacrylamide and N-alkyl-substituted acrylamides having linear, branched-chain or carbocyclic alkyl radicals, where the alkyl radical can have the meanings given above for R⁴.

Monomers (e), which have a crosslinking function, are compounds having at least two ethylenically unsaturated nonconjugated double bonds in the molecule.

Suitable crosslinkers are, for example, acrylic esters, methacrylic esters, allyl ethers or vinyl ethers of at least dihydric alcohols. The OH groups of the parent alcohols can be completely or partially etherified or esterified; however, the crosslinkers contain at least two ethylenically unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-yclohexanediol, 1,4-cyclohexanediol, 1,4-bis(hydroxymethyl)cyclohexane, mononeopentyl glycol hydroxypivalate, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and also polyethylene glycols, polypropylene glycols and polytetrahydrofurans each having molecular weights of from 200 to 10,000. As well as the homopolymers of ethylene oxide or propylene oxide, it is also possible to use block copolymers of ethylene oxide or propylene oxide or copolymers which contain incorporated ethylene oxide and propylene oxide groups. Examples of parent alcohols having more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose and mannose. It is of course also possible to use the polyhydric alcohols following reaction with ethylene oxide or propylene oxide as the corresponding ethoxylates or propoxylates respectively. The polyhydric alcohols can also be firstly converted into the corresponding glycidyl ethers by reaction with epichlorohydrin.

Other suitable crosslinkers are the vinyl esters or the esters of monohydric, unsaturated alcohols with ethylenically unsaturated C₃- to C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. It is, however, also possible to esterify the monohydric, unsaturated alcohols using polybasic carboxylic acids, for example malonic acid, tartaric acid, trimellitic acid, phthalic acid, terephthalic acid, citric acid or succinic acid.

Other suitable crosslinkers are esters of unsaturated carboxylic acids with the above-described-polyhydric alcohols, for example oleic acid, crotonic acid, cinnamic acid or 10-undecanoic acid.

Also suitable as monomers (e) are straight-chain or branched, linear or cyclic, aliphatic or aromatic hydrocarbons which have at least two double bonds, which in the case of aliphatic hydrocarbons must not be conjugated, e.g. divinyl benzene, divinyl toluene, 1,7-octadiene, 1,9-decadiene, 4-vinyl-1-cyclohexene, trivinylcyclohexane or polybutadienes having molecular weights from 200 to 20,000.

Other suitable crosslinkers are acrylamides, methacrylamides and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Also suitable are the amides of allylamine and unsaturated carboxylic acids such as acrylic acid, methacrylic acid, itaconic acid, maleic acid, or at least dibasic carboxylic acids as described above.

Other suitable crosslinkers are triallylamine and triallylmonoalkylammonium salts, e.g. triallylmethylammonium chloride or methylsulfate.

Other suitable crosslinkers are N-vinyl compounds of urea derivatives, at least difunctional amides, cyanurates or urethanes, for example of urea, ethyleneurea, propyleneurea, or tartramide, e.g. N,N′-divinylethyleneurea or N,N′-divinylpropyleneurea.

Further suitable crosslinkers are divinyldioxane, tetrallylsilane or tetravinylsilane.

Preference is given to using crosslinkers which are soluble in the monomer mixture.

Particularly preferred crosslinkers are, for example, pentaerythritol triallyl ether, methylenebisacrylamide, triallylamine and triallylalkylammonium salts, divinylimidazole, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin.

Very particularly preferred crosslinkers are pentaerythritol triallyl ether, methylenebisacrylamide, N,N′-divinylethyleneurea, triallylamine and acrylic esters of glycol, butanediol, trimethylolpropane or glycerol or acrylic esters of glycol, butanediol, trimethylolpropane or glycerol reacted with ethylene oxide and/or epichlorohydrin.

The monomers (a) to (e) can each be used individually or in a mixture with other monomers of the same group.

The polymers are prepared by the processes of free-radically-initiated solution polymerization known per se, preferably in aqueous media, particularly preferably in water without the addition of a further solvent.

The polymerization is usually carried out at temperatures of from 20° C. to 150° C. and at atmospheric pressure or under autogenous pressure; the temperature can be kept constant or be increased continuously or discontinuously, e.g. in order to increase the conversion.

Initiators which can be used for the free-radical polymerization are the water-soluble and water-insoluble peroxo and/or azo compounds customary for this purpose, for example alkali metal or ammonium peroxodisulfates, dibenzoyl peroxide, tert-butyl perpivalate, tert-butyl per-2-ethylhexanoate, di-tert-butyl peroxide, tert-butyl hydroperoxide, azobisisobutyronitrile, azobis(2-amidinopropane) dihydrochloride or 2,2′-azobis(2-methylbutyronitrile). Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate. The initiators can be used in the customary amounts, for example, 0.05 to 5% by weight, or 0.05 to 0.3 mol %, based on the amount of monomers to be polymerized.

Polymerization results in a solution having a solids content of from 5 to 40% by weight, preferably from 5 to 35% by weight, particularly preferably from 7 to 20% by weight. To increase the solids content, the solution can be partially or completely dewatered by distillation.

The crosslinked or noncrosslinked polymers prepared can be used directly in skin cosmetic or dermatological applications. The polymers are not isolated, but-are preferably used directly in the form of their solution.

The molecular weight and the K value of the copolymers used according to the invention can be varied within a wide range in a manner known per se through the choice of polymerization conditions, for example polymerization time, polymerization temperature or initiator concentration, and by the content of crosslinker. The K values of preferred polymers are in a range between 30 and 350, preferably 50 and 350.

The K values are measured in accordance with Fikentscher, Cellulosechemie, Vol. 13, pp. 58-64 (1932) at 25° C. at 0.1% strength in 0.5 molar sodium chloride solution.

In the case of high degrees of crosslinking, the K values of the polymers cannot be determined.

The inorganic UV filters used in the mixtures according to the invention are, for example, inorganic pigments based on metal oxides and/or other metal compounds which are insoluble or sparingly soluble in water, preferably the oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals and mixtures of such oxides. The pigments are particularly preferably based on TiO₂ or ZnO, very particularly preferably on ZnO, in particular are ZnO particles as in EP-A-585 239 having a mean particle diameter of less than 0.2 μm, which contain less than 20 ppm of lead, less than 3 ppm of arsenic, less than 15 ppm of cadmium and less than 1 ppm of mercury.

The inorganic pigments are advantageously in hydrophobic form, i.e. have been surface-treated to repel water. This surface treatment can involve providing the pigments with a thin hydrophobic layer, in particular a layer of silicone, by methods known per se.

One such method consists, for example, in producing the hydrophobic surface layer according to the reaction in accordance with

nTiO₂+m(RO)₃Si—R′→nTiO₂ (surf.).

n and m are stoichiometric parameters to be used as desired, and R and R′ are the desired organic radicals. Examples which may be mentioned are the hydrophobicized pigments prepared analogously to DE-A-33 14 742.

In a preferred embodiment, the metal oxide is coated with a silicone of the formula III

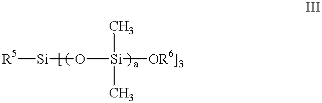

in which, independently of one another, R⁵ is C₁-C₁₂-alkyl, preferably octyl, butyl or ethyl, and R⁶ is methyl or ethyl, and a is a value from 4 to 12, preferably 4 to 8. With regard to the preparation and properties of these silicone-coated metal oxides, reference is made to U.S. Pat. No. 5,756,788.

The proportion of inorganic UV filters in the mixture according to the invention is 0.1 to 99.9% by weight, preferably 10 to 95% by weight, particularly preferably 30 to 90% by weight, based on the solids content of the mixture.

It is also advantageous to add further oil-soluble and/or water-soluble organic UV-A and/or UV-B filters to the mixture according to the invention, the total amount of organic filter substances being, for example, 1 to 300% by weight, preferably 10 to 250% by weight, particularly preferably 50 to 200% by weight, based on the solids content of the mixture.

Examples thereof are:

TABLE 1

| | | CAS No. |
|---|---|---|
| No. | Substance | (= acid) |
| 1 | 4-aminobenzoic acid | 150-13-0 |
| 2 | 3-(4′-trimethylammonium)benzylidenebornan-2-one | 52793-97-2 |
| | methylsulfate | |
| 3 | 3,3,5-trimethylcyclohexyl salicylate | 118-56-9 |
| | (homosalate) | |
| 4 | 2-hydroxy-4-methoxybenzophenone | 131-57-7 |
| | (oxybenzone) | |
| 5 | 2-phenylbenzimidazole-5-sulfonic acid and its | 27503-81-7 |
| | potassium, sodium and triethanolamine salts | |
| 6 | 3,3′-(1,4-phenylenedimethine)bis(7,7-dimethyl- | 90457-82-2 |
| | 2-oxobicyclo[2.2.1]heptane-1-methanesulfonic | |
| | acid) and its salts | |
| 7 | polyethoxyethyl 4-bis(polyethoxy)aminobenzoate | 113010-52-9 |
| 8 | 2-ethylhexyl 4-dimethylaminobenzoate | 21245-02-3 |
| 9 | 2-ethylhexyl salicylate | 118-60-5 |
| 10 | 2-isoamyl 4-methoxycinnamate | 71617-10-2 |
| 11 | 2-ethylbexyl 4-methoxycinnamate | 5466-77-3 |
| 12 | 2-hydroxy-4-methoxybenzophenone-5-sulfonic acid | 4065-45-6 |
| | (sulisobenzone) and the sodium salt | |
| 13 | 3-(4′-sulfobenzylidene)bornan-2-one and salts | 58030-58-6 |
| 14 | 3-benzylidenebornan-2-one | 16087-24-8 |
| 15 | 1-(4′-isopropylphenyl)-3-phenylpropane-1,3- | 63260-25-9 |
| | dione | |
| 16 | 4-isopropylbenzyl salicylate | 94134-93-7 |
| 17 | 2,4,6-trianilino(o-carbo-2′-ethylhexyl-1′-oxy)- | 88122-99-0 |
| | 1,3,5-triazine | |
| 18 | 3-imidazol-4-ylacrylic acid and its ethyl ester | 104-98-3 |
| 19 | menthyl o-aminobenzoate or: | 134-09-8 |
| | 5-methyl-2-(1-methylethyl)-2-aminobenzoate | |
| 20 | glyceryl p-aminobenzoate or: | 136-44-7 |
| | 1-glyceryl 4-aminobenzoate | |
| 21 | 2,2′-dihydroxy-4-methoxybenzophenone | 131-53-3 |
| | (dioxybenzone) | |
| 22 | 2-hydroxy-4-methoxy-4-methylbenzophenone | 1641-17-4 |
| | (mexenone) | |
| 23 | triethanolamine salicylate | 2174-16-5 |
| 24 | dimethoxyphenylglyoxalic acid or: | 4732-70-1 |
| | sodium 3,4-dimethoxyphenylglyoxalate | |
| 25 | 3-(4′-sulfobenzylidene)bornan-2-one and its | 56039-58-8 |
| | salts | |
| 26 | 2,2′,4,4′-tetrahydroxybenzophenone | 131-55-5 |
| 27 | 2,2′-methylenebis[6-(2H-benzotriazol-2-yl)-4- | 103597-45-1 |
| | (1,1,3,3-tetramethylbutyl)phenol] | |
| 28 | 2,2′-(1,4-phenylene)bis-1H-benzimidazole-4,6- | 180898-37-7 |
| | di-sulfonic acid, Na salt | |
| 29 | 2,4-bis[4-(2-ethylhexyloxy)-2-hydroxy]phenyl-6- | 187393-00-6 |
| | (4-methoxyphenyl)-(1,3,5) triazine | |
| 30 | 3-(4-methylbenzylidene) camphor | 36861-47-9 |
| 31 | polyethoxyethyl | 113010-52-9 |
| | 4-bis(polyethoxy)paraaminobenzoate | |
| 32 | 2,4-dihydroxybenzophenone | 131-56-6 |
| 33 | 2,2′-dihydroxy-4,4′-dimethoxybenzophenone-5,5′- | 3121-60-6 |
| | disodium sulfonate | |

Other sunscreens which can be combined are, inter alia, the compounds below described in WO 94/05645 and EP-A-0 444 323:

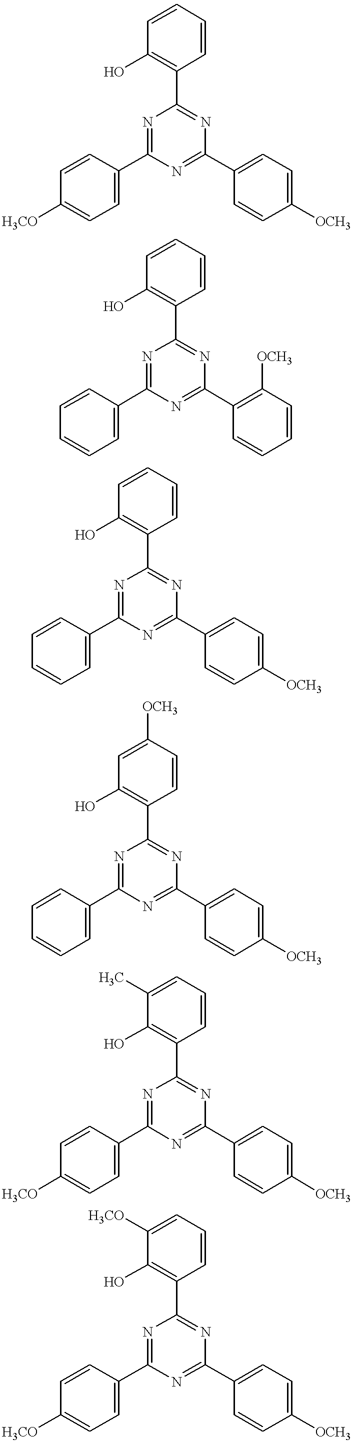

The list of UV filters given, which can be used in combination with the active ingredient combinations according to the invention, is of course not intended to be limiting.

The mixtures according to the invention are suitable as starting materials for the preparation of cosmetic and dermatological preparations.

The invention therefore also relates in particular to the use of the mixtures mentioned in the introduction as photostable UV filters in cosmetic and dermatological preparations for protecting human skin or human hair against solar rays, alone or together with compounds which absorb in the UV region and are known per se for cosmetic and pharmaceutical preparations.

Preference is given to the use in skincare compositions, sunscreens, anti-acne agents, foundation, mascara, lipsticks, eyeshadows, kohl pencils, eyeliners, blushers, powders and eyebrow pencils.

The invention further relates to cosmetic and dermatological sunscreen preparations for protecting the human skin or human hair against solar rays, comprising the mixture mentioned at the outset.

The skincare or sunscreen preparations are in particular in the form of W/O or O/W skin creams, day and night creams, eye creams, face creams, antiwrinkle creams, moisturizing creams, bleaching creams, vitamin creams, skin lotions, care lotions and moisturizing lotions.

In the cosmetic and dermatological preparations, the mixtures according to the invention can display particular effects. In addition to the UV ray-absorbing/reflecting properties of the inorganic UV filters (component B), the polymers (component A) can inter alia contribute to the moisturizing and conditioning of the skin and to improving the feel of the skin. By adding the polymers according to the invention, a considerable improvement in skin compatibility can be achieved in certain formulations.

The polymers (A) further effect stabilization of cosmetic and dermatological preparations, in particular of emulsions which comprise pigments [lacuna] inorganic UV absorbers.

A further advantage of the mixtures according to the invention is the attainment of relatively high sun protection factors in the cosmetic and dermatological formulations. For the same amount of inorganic UV filters (B) used, the addition of the cationic polymers (A) leads to an increase in the sun protection factor by a factor of at least 1.1 to 3.0, preferably 1.1 to 2.0, particularly preferably 1.2 to 1.5, compared with preparations without (A).

The mixtures according to the invention are present in the skin cosmetic and dermatological preparations in an amount of from about 0.001 to 30% by weight, preferably 0.01 to 25% by weight, particularly preferably 0.1 to 20% by weight, very particularly preferably 1 to 15% by weight, based on the total weight of the preparation.

In addition to the mixtures according to the invention and suitable solvents, the skin cosmetic preparations can also comprise additives customary in cosmetics, such as emulsifiers, preservatives, perfume oils, cosmetic active ingredients, such as phytantriol, vitamin A, E and C, retinol, bisabolol, panthenol, bleaches, colorants, tinting agents, tanning agents (e.g. dihydroxyacetone), collagen, protein hydrolysates, stabilizers, pH regulators, dyes, salts, thickeners, gel formers, bodying agents, silicones, moisturizers, refatting agents and other customary additives.

Suitable solvents which can be mentioned are, in particular, water and lower monoalcohols or polyols having 1 to 6 carbon atoms or mixtures thereof; preferred monoalcohols or polyols are ethanol, isopropanol, propylene glycol, glycerol and sorbitol.

An additional content of antioxidants is generally preferred. According to the invention, favorable antioxidants may be all antioxidants which are customary or suitable for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximine, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-silbene oxide) and the derivatives (salts, esters, ethers, sugars, nucleotides, nucleosides, peptides and lipids) of said active ingredients which are suitable according to the invention.

The amount of the abovementioned antioxidants (one or more compounds) in the preparations is preferably 0.001 to 30% by weight, particularly preferably 0.05 to 20% by weight, in particular 1 to 10% by weight, based on the total weight of the preparation.

Customary thickeners in such formulations are crosslinked polyacrylic acids and derivatives thereof, polysaccharides, such as xanthan gum, agar agar, alginates or Tyloses, carboxymethylcellulose or hydroxycarboxymethylcellulose, fatty alcohols, monoglycerides and fatty acids, polyvinyl alcohol and polyvinylpyrrolidone.

The mixtures according to the invention can also be mixed with conventional polymers if specific properties are to be set.

Examples of suitable conventional polymers are anionic, cationic, amphoteric and neutral polymers.

Examples of anionic polymers are homo- and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of t-butyl acrylate, ethyl acrylate, methacrylic acid (e.g. Luvimer® 100P), copolymers of ethyl acrylate and methacrylic acid (e.g. Luvimer® MAE), copolymers of N-tert-butylacrylamide, ethyl acrylate, acrylic acid (Ultrahold® 8, strong), copolymers of vinyl acetate, crotonic acid and optionally other vinyl esters (e.g. Luviset® grades), maleic anhydride copolymers, optionally reacted with alcohols, anionic polysiloxanes, e.g. carboxy-functional ones, copolymers of vinylpyrrolidone, t-butyl acrylate, methacrylic acid (e.g. LuviskolO VBM), copolymers of acrylic acid and methacrylic acid with hydrophobic monomers, such as, for example, C₄-C₃₀-alkyl esters of (meth)acrylic acid, C₄-C₃₀-alkylvinyl esters, C₄-C₃₀-alkyl vinyl ethers and hyaluronic acid.

Further suitable polymers are also neutral polymers, such as polyvinylpyrrolidones, copolymers of N-vinylpyrrolidone and vinyl acetate and/or vinyl propionate, polysiloxanes, polyvinylcaprolactam and copolymers containing N-vinylpyrrolidone, polyethyleneimines and salts thereof, polyvinylamines and salts thereof, cellulose derivatives, polyaspartic acid salts and derivatives.

To set certain properties, the preparations can additionally also comprise conditioning substances based on silicone compounds. Suitable silicone compounds are, for example, polyalkylsiloxanes, polyarylsiloxanes, polyarylalkylsiloxanes, polyether siloxanes or silicone resins.

The mixtures according to the invention are used in cosmetic or dermatological preparations, the preparation of which is carried out in accordance with the customary principles familiar to the person skilled in the art.

Such formulations are advantageously in the form of emulsions, preferably as water-in-oil (W/O) or oil-in-water (O/W) emulsions. According to the invention, it is, however, also possible and in some cases advantageous to choose other types of formulation, for example hydrodispersions, gels, oils, oleogels, multiple emulsions, for example in the form of W/O/W or O/W/O emulsions, anhydrous ointments or ointment bases etc.

The emulsions which can be used according to the invention are prepared by known methods.

In addition to the mixtures according to the invention, the emulsions comprise customary constituents, such as fatty alcohols, fatty acid esters and in particular fatty acid triglycerides, fatty acids, lanolin and derivatives thereof, natural or synthetic oils or waxes and emulsifiers in the presence of water.

The choice of emulsion-type-specific additives and the preparation of suitable emulsions is described, for example, in Schrader, Grundlagen und Rezepturen der Kosmetika [Cosmetic bases and formulations], Hüthig Buch Verlag, Heidelberg, 2^{nd}Edition, 1989, third part, to which reference is expressly made here.

Thus, a skin cream which can be used according to the invention can, for example, be in the form of a W/O emulsion. An emulsion of this type comprises an aqueous phase which is emulsified in an oil or fatty phase using a suitable emulsifier system.

The concentration of the emulsifier system in this type of emulsion is between about 4 and 35% by weight, based on the total weight of the emulsion; the fatty phase constitutes between about 20 and 60% by weight and the aqueous phase between about 20 and 70% by weight, in each case based on the total weight of the emulsion. The emulsifiers are those which are customarily used in this type of emulsion. They are chosen, for example, from: C₁₂-C₁₈-sorbitan fatty acid esters; esters of hydroxystearic acid and C₁₂-C₃₀-fatty alcohols; mono- and diesters of C₁₂-C₁₈-fatty acids and glycerol or polyglycerol; condensates of ethylene oxide and propylene glycols; oxypropylenated/oxyethylenated C₁₂-C₂₀-fatty alcohols; polycyclic alcohols, such as sterols; aliphatic alcohols having a high molecular weight, such as lanolin; mixtures of oxypropylenated/polyglycerolated alcohols and magnesium isostearate; succinic esters of polyoxyethylenated or polyoxypropylenated fatty alcohols; and mixtures of magnesium, calcium, lithium, zinc or aluminum lanolate and hydrogenated lanolin or lanolin alcohol.

Suitable fatty components which may be present in the fatty phase of the emulsions include hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karite oil, hoplostethus oil, mineral oils whose distillation start point under atmospheric pressure is at about 250° C. and whose distillation end point is at 410° C., such as, for example, vaseline oil; esters of saturated or unsaturated fatty acids, such as alkyl myristates, e.g. isopropyl, butyl or cetyl myristate, hexadecyl stearate, ethyl or isopropyl palmitate, octanoic or decanoic triglycerides and cetyl ricinoleate.

The fatty phase can also comprise silicone oils which are soluble in other oils, such as dimethylpolysiloxane, methylphenylpolysiloxane and the silicone glycol copolymer, fatty acids and fatty alcohols.

In order to promote the retention of oils, it is also possible to use waxes, such as, for example, carnauba wax, candellila wax, beeswax, microcrystalline wax, ozokerite wax and Ca, Mg and Al oleates, myristates, linoleates and stearates.

These water-in-oil emulsions are generally prepared by adding the fatty phase and the emulsifier to the charging container. These are then heated at a temperature of from 70 to 75° C., then the oil-soluble ingredients are added and, with stirring, water is added which has been heated beforehand to the same temperature and in which the water-soluble ingredients have been dissolved beforehand; the mixture is stirred until an emulsion of the desired fineness is obtained, which is then left to cool to room temperature, if necessary with gentle stirring.

A care emulsion according to the invention can also be in the form of a O/W emulsion. An emulsion of this type usually comprises an oil phase, emulsifiers which stabilize the oil phase in the water phase, and an aqueous phase, which is usually in thickened form.

The aqueous phase of the O/W emulsion of the preparations according to the invention optionally comprises

alcohols, diols or polyols, and ethers thereof, preferably ethanol, isopropanol, propylene glycol, glycerol, ethylene glycol monoethyl ether;

customary thickeners or gel formers, such as, for example, crosslinked polyacrylic acids and derivatives thereof, polysaccharides, such as xanthan gum or alginates, carboxymethylcellulose or hydroxycarboxymethylcellulose, fatty alcohols, polyvinyl alcohol and polyvinylpyrrolidone.

The preparation can be carried out by melting the oil phase at about 80° C.; the water-soluble constituents are dissolved in hot water, and added slowly and with stirring to the oil phase; the mixture is then homogenized and stirred until cold.

### A PREPARATION OF THE POLYMERS

### Preparation Example 1

A stirred apparatus was charged with 400 g of water and 46 g of dimethyldiallylammonium chloride solution (65% strength). 10% of Feed 1, consisting of 270 g of N-vinylpyrrolidone and 0.6 g of N,N′-divinylethyleneurea, was added to this initial charge. The mixture was heated to 60° C. with stirring in a stream of nitrogen, and Feed 1 was metered in over the course of 3 hours, and Feed 2, consisting of 0.9 g of 2,2′-azobis(2-amidinopropane) dihydrochloride in 100 g of water, was metered in over the course of 4 hours. After 3 hours, the mixture was diluted with 700 g of water and stirred for a further hour. Then, 1.5 g of 2,2′-azobis(2-amidinopropane) dihydrochloride in 30 g of water were added and the mixture was stirred for a further 2 hours at 60° C. This gave a colorless high-viscosity polymer solution with a solids content of 20.9% and a K value of 80.3.

### Preparation Example 2

A stirred apparatus was charged with 300 g of Feed 1, consisting of 200 g of N-vinylpyrrolidone, 77 g of dimethyldiallylammonium chloride solution (65% strength), 1.13 g of N,N′-divinylethyleneurea and 440 g of water, and the mixture was heated to 60° C. with stirring and in a stream of nitrogen. The remainder of Feed 1 was metered in over 2 hours, and Feed 2, consisting of 0.75 g of 2,2′-azobis(2-amidinopropane) dihydrochloride in 100 g of water, was metered in over 4 hours. When the addition of Feed 1 was complete, the reaction mixture was diluted with 1620 g of water. When the addition of Feed 2 was complete, the mixture was stirred for a further hour at 60° C., then 1.25 g of 2,2′-azobis(2-amidinopropane) dihydrochloride in 65 g of water were added and the mixture was stirred for a further hour. This gave a colorless high-viscosity polymer solution with a solids content of 10.2% and a K value of 80.

### Preparation Example 3

A stirred apparatus was charged with 130 g of water and 48 g of 3-methyl-1-vinylimidazolium chloride, and the mixture was heated to 60° C. with stirring and under a stream of nitrogen. Then, Feed 1, consisting of 192 g of N-vinylpyrrolidone, 0.48 g of N,N′-divinylethyleneurea and 450 g of water, was metered in over 3 hours, and Feed 2, consisting of 1.44 g of 2,2′-azobis(2-amidinopropane) dihydrochloride in 80 g of water, was metered in over 4 hours. The mixture was then stirred for a further hour at 60° C. In order to keep the mixture stirrable, it was diluted with a total of 2100 g of water as required. This gave a colorless high-viscosity polymer solution with a solids content of 8.2% and a K value of 105.

### Preparation Example 4

716 g of water were charged to a stirred apparatus and, with stirring and under a stream of nitrogen, heated to 60° C. Then, Feed 1, consisting of 180 g of N-vinylpyrrolidone, 20 g of 3-methyl-1-vinylimidazolium methylsulfate, 0.32 g of N,N′-divinylethyleneurea and 25 g of water, was metered in over 2 hours, and Feed 2, consisting of 0.6 g of 2,2′-azobis(2-amidinopropane) dihydrochloride in 60 g of water, was metered in over 3 hours. When the addition of Feed 1 was complete, the reaction mixture was diluted with 1000 g of water. Following the addition of Feed 2, the mixture was stirred for a further 3 hours at 70° C. This gave a colorless high-viscosity polymer solution with a solids content of 11.0% and a K value of 86.

### Preparation Example 5

440 g of water were charged to a stirred apparatus and, with stirring and in a stream of nitrogen, heated to 60° C. Then, Feed 1, consisting of 180 g of N-vinylpyrrolidone, 20 g of 3-methyl-1-vinylimidazolium methylsulfate, 0.30 g of N,N′-divinylethyleneurea and 25 g of water, was metered in over 2 hours, and Feed 2, consisting of 0.6 g of 2,2′-azobis(2-amidinopropane) dihydrochloride in 60 g of water, was metered in over 3 hours. Following the addition of Feed 2, the mixture was stirred for a further 3 hours at 70° C. In order to keep the reaction mixture stirrable, it was diluted with a total of 1275 g of water as required. This gave a colorless high-viscosity polymer solution with a solids content of 11.3% and a K value of 105.

### Preparation Example 6

650 g of water were charged to a stirred apparatus and, with stirring and in a stream of nitrogen, heated to 60° C. Then, Feed 1, consisting of 225 g of N-vinylpyrrolidone, 25 g of 2,3-dimethyl-1-vinylimidazolium methylsulfate, 0.25 g of N,N′-divinylethyleneurea and 580 g of water, was metered in over 3 hours, and Feed 2, consisting of 0.7 g of 2,2′-azobis(2-amidinopropane) dihydrochloride in 100 g of water, was metered in over 4 hours. When the addition of Feed 1 was complete, the reaction mixture was diluted with 835 g of water. Following the addition of Feed 2, the mixture was stirred for a further hour, and 1.25 g of 2,2′-azobis(2-amidinopropane) dihydrochloride in 77 g of water were then metered in. The mixture was then stirred for a further 2 hours at 70° C. This gave 35 a colorless high-viscosity polymer solution with a solids content of 10.4% and a K value of 106.

### Preparation Example 7

650 g of water were charged to a stirred apparatus and, with stirring and in a stream of nitrogen, heated to 60° C. Then, Feed 1, consisting of 225 g of N-vinylpyrrolidone, 25 g of 2,3-dimethyl-1-vinylimidazolium methylsulfate, 0.375 g of N,N′-divinylethyleneurea and 580 g of water, was metered in over 3 hours, and Feed 2, consisting of 0.7 g of 2,2′-azobis(2-amidinopropane) dihydrochloride in 100 g of water, were metered in over 4 hours. When the addition of Feed 1 was complete, the reaction mixture was diluted with 1135 g of water. Following the addition of Feed 2, the mixture was stirred for a further hour, and 1.25 g of 2,2′-azobis(2-amidinopropane) dihydrochloride in 77 g of water were then metered in. The mixture was then stirred for a further 2 hours at 70° C. This gave a colorless high-viscosity polymer solution with a solids content of 9.2% and a K value of 92.

### Preparation Example 8

440 g of water were charged to a stirred apparatus and, with stirring and in a stream of nitrogen, heated to 60° C. Then, Feed 1, consisting of 144 g of N-vinylpyrrolidone, 16 g of 3-methyl-1-vinylimidazolium methylsulfate, 1.4 g of tetraethylene glycol diacrylate and 100 g of water, was metered in over 2 hours, and Feed 2, consisting of 0.8 g of 2,2′-azobis(2-amidinopropane) dihydrochloride in 50 g of water, was metered in over 3 hours. Following the addition of Feed 2, the mixture was stirred for a further 3 hours at 70° C. In order to keep the reaction mixture stirrable, it was diluted with a total of 1200 g of water as required. This gave a colorless high-viscosity polymer solution with a solids content of 8.5% and a K value of 95.

### Preparation Example 9

550 g of water were charged to a stirred apparatus and, with stirring and in a stream of nitrogen, heated to 60° C. Then, Feed 1, consisting of 102 g of N-vinylpyrrolidone, 26 g of 3-methyl-1-vinylimidazolium methylsulfate, 0.8 g of triallylamine and 100 g of water, was metered in over 2 hours. Feed 2, consisting of 0.6 g of 2,2′-azobis(2-amidinopropane) dihydrochloride in 50 g of water, was added to the reaction mixture over 3 hours. Following the addition of Feed 2, the mixture was stirred for a further 3 hours at 70° C. In order to keep the reaction mixture stirrable, it was diluted with a total of 1000 g of water as required. This gave a pale yellowish high-viscosity polymer solution with a solids content of 7.0% and a K value of 102.

### B APPLICATION EXAMPLES

### Application Example 1

Sunscreen cream (A)

Firstly, a water/oil cream emulsion (sunscreen cream A) according to the invention was prepared in accordance with the following recipe:

| | % by |
|---|---|
| Additive | weight |
| Ceteareth-6 and stearyl alcohol | 1.0 |
| Ceteareth-25 | 2.0 |
| Glyceryl stearate | 3.0 |
| Cetearyl alcohol | 2.0 |
| Cetearyl octanoate | 2.0 |
| Uvinul T150 (octyltriazone) | 1.0 |
| Uvinul MC 80 (octyl methoxycinnamate) | 5.0 |
| Uvinul MBC 95 (4-methylbenzylidenecamPhor) | 3.0 |
| Z-Cote HP-1 (zinc oxide) | 5.0 |
| Isopropyl myristate | 7.0 |
| D-panthenol | 0.5 |
| 1,2-propylene glycol | 5.0 |
| Polymer (Preparation Example 9) | 0.5 |
| Xanthan gum (2% in water) | 15.0 |
| Tocopherol acetate | 1.0 |
| Perfume oil | q.s. |
| Preservative | q.s. |
| Water | ad 100 |

Sun protection factor: 20 (determined in accordance with the Colipa method, described in Parfuem. Kosmet. (1994), 75(12), 856)

### Comparative Example 1

Sunscreen cream (B)—without the addition of polymer

| | % by |
|---|---|
| Additive | weight |
| Ceteareth-6 and stearyl alcohol | 1.0 |
| Ceteareth-25 | 2.0 |
| Glyceryl stearate | 3.0 |
| Cetearyl alcohol | 2.0 |
| Cetearyl octanoate | 2.0 |
| Uvinul T150 (octyltriazone) | 1.0 |
| Uvinul MC 80 (octyl methoxycinnamate) | 5.00 |
| Uvinul MBC 95 (4-methylbenzylidenecamphor) | 3.00 |
| Z-Cote HP-1 (zinc oxide) | 5.00 |
| Isopropyl myristate | 7.00 |
| D-panthenol | 0.50 |
| 1,2-propylene glycol | 5.0 |
| Polymer (Preparation Example 9) | — |
| Xanthan gum (2% in water) | 15.00 |
| Tocopherol acetate | 1.00 |
| Perfume oil | q.s. |
| Preservative | q.s. |
| Water | ad 100 |

Sun protection factor: 15 (determined in accordance with the Colipa method, described in Parfuem. Kosmet. (1994), 75(12), 856)

### Application Example 2

Sunscreen cream (C)

Firstly, a water/oil cream emulsion (sunscreen cream C) according to the invention was prepared in accordance with the following recipe:

| | % by |
|---|---|
| Additive | weight |
| Ceteareth-6 and stearyl alcohol | 1.0 |
| Ceteareth-25 | 2.0 |
| Glyceryl stearate | 4.0 |
| Cetearyl alcohol | 2.0 |
| Cetearyl octanoate | 2.0 |
| Uvinul T150 (octyltriazone) | 1.0 |
| Uvinul MC 80 (octyl methoxycinnamate) | 5.0 |
| Uvinul MBC 95 (4-methylbenzylidenecamphor) | 3.0 |
| Z-Cote HP-1 (zinc oxide) | 5.0 |
| Isopropyl myristate | 7.0 |
| Dimethicone | 1.0 |
| D-panthenol | 0.5 |
| 1,2-propylene glycol | 5.0 |
| Polymer (Preparation Example 9) | 0.5 |
| EDTA | 0.2 |
| Tocopherol acetate | 1.0 |
| Phenoxyethanol | 0.5 |
| Methyldibromoglutaronitrile | q.s. |
| Water | ad 100 |

The formulation was colloidally stable

### Comparative Example 2

Sunscreen cream (D)—without the addition of Polymer

| | % by |
|---|---|
| Additive | weight |
| Ceteareth-6 and stearyl alcohol | 1.0 |
| Ceteareth-25 | 2.0 |
| Glyceryl stearate | 4.0 |
| Cetearyl alcohol | 2.0 |
| Cetearyl octanoate | 2.0 |
| Uvinul T150 (octyltriazone) | 1.0 |
| Uvinul MC 80 (octyl methoxycinnamate) | 5.0 |
| Uvinul MBC 95 (4-methylbenzylidenecamphor) | 3.0 |
| Z-Cote HP-1 (zinc oxide) | 5.0 |
| Isopropyl myristate | 7.0 |
| Dimethicone | 1.0 |
| D-panthenol | 0.5 |
| 1,2-propylene glycol | 5.0 |
| Polymer (Preparation Example 9) | — |
| EDTA | 0.2 |
| Tocopherol acetate | 1.0 |
| Phenoxyethanol | 0.5 |
| Methyldibromoglutaronitrile | q.s. |
| Water | ad 100 |

The formulation was colloidally unstable

We claim:

1. A mixture comprising
A) at least one copolymer obtainable by
(i) free-radically initiated solution polymerization of a monomer mixture of
(a) 0.01 to 99.99% by weight of at least one monomer chosen from the group consisting of N-vinylimidazoles and diallylamines, optionally in partially or completely quaternized form;
(b) 0.01 to 99.99% by weight of at least one neutral or basic water-soluble monomer which is different from (a);
(c) 0 to 50% by weight of at least one unsaturated acid or an unsaturated anhydride;
(d) 0 to 50% by weight of at least one free-radically copolymerizable monomer which is different from (a), (b) and (c); and
(e) 0 to 10% by weight of at least one monomer having at least two ethylenically unsaturated nonconjugated double bonds which acts as crosslinker, and
(ii) subsequent partial or complete quaternization or protonation of the polymer where the monomer (a) is not quaternized or only partially quaternized and
B) at least one inorganic UV filter.

2. A mixture as claimed in claim 1, wherein the copolymer A) is obtainable by solution polymerization in water.

3. A mixture as claimed in either of claims 1 and 2, wherein the monomer (e) used is 0.01 to 10% by weight of at least one monomer having at least two ethylenically unsaturated nonconjugated double bonds which acts as crosslinker.

4. A mixture as claimed in any of claims 1 to 3, wherein the protonation according to (ii) takes place during the preparation of the mixture.

5. A mixture as claimed in any of claims 1 to 4, wherein the monomer (a) used is at least one N-vinylimidazole derivative of the formula (I) 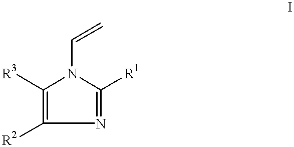
in which the radicals R¹ to R³, independently of one another, are hydrogen, C₁-C₄-alkyl or phenyl.

6. A mixture as claimed in any of claims 1 to 4, wherein the monomer (a) used is at least one diallylamine derivative of the formula (II) 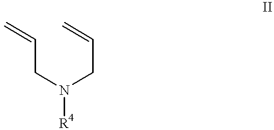
in which the radical R⁴ is C₁-C₂₄-alkyl.

7. A mixture as claimed in any of claims 1 to 6, wherein the monomer (b) used is at least one N-vinyllactam.

8. A mixture as claimed in any of claims 1 to 7, comprising, as inorganic UV filter B), at least one micronized metal oxide chosen from the group consisting of titanium dioxide, zinc oxide, cerium oxide, aluminum oxide, silicon oxide, zirconium oxide, manganese oxide, aluminum oxide and iron oxide.

9. A mixture as claimed in claim 8, comprising, as inorganic UV filter B), at least one hydrophobicized metal oxide chosen from the group consisting of titanium dioxide and zinc oxide.

10. A mixture as claimed in claim 9, in which the metal oxide has been coated with a silicone of the formula III 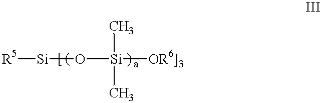
in which, independently of one another, R⁵ is C₁-C₁₂-alkyl and R⁶ is methyl or ethyl, and a is a value from 4 to 12.

11. A mixture as claimed in any of claims 1 to 10, wherein the proportion of inorganic UV filters is 0.1 to 99.9% by weight.

12. A mixture as claimed in any of claims 1 to 11, comprising at least one further organic UVA and/or UVB filter.

13. The use of a mixture defined as in any of claims 1 to 12 for the preparation of cosmetic and dermatological preparations.

14. The use as claimed in claim 13 as photostable UV filter in cosmetic and dermatological preparations for protecting the human skin or human hair against solar rays, alone or together with compounds which absorb in the UV region and which are known per se for cosmetic and pharmaceutical preparations.

15. A cosmetic or dermatological sunscreen preparation for protecting the human skin or human hair against solar rays, comprising a mixture defined as in any of claims 1 to 12.
